(19) United States
(12) Patent Application Publication
(10) Pub. No.: US 2007/0269900 A1
(43) Pub. Date: Nov. 22, 2007

(54) Use of Conductive or Semiconductive Polymers in Chemical Sensors for the Detection of Nitro Compounds

(75) Inventors: Bruno Lebret, Chambray-Les-Tours (FR); Lionel Hairault, Blere (FR); Eric Pasquinet, Saint Avertion (FR); Pierrick Buvat, Mont Razon (FR)

Correspondence Address: MCKENNA LONG & ALDRIDGE LLP, 1900 K STREET, NW, WASHINGTON, DC, 20006, US

(73) Assignee: Commissariat A L'Energie Atomique, Paris (FR)

(21) Appl. No.: 10/576,365

(22) PCT Filed: Oct. 19, 2004
(86) PCT No.: PCT/FR04/02670

§ 371 Date: Mar. 14, 2007

(30) Foreign Application Priority Data
Oct. 20, 2003 (FR) 0350709

(51) Int. Cl. G01N 33/00; G01N 21/64; H01B 1/12; G01N 27/12

(52) U.S. Cl. 436/107; 436/106

(57) ABSTRACT

The invention relates to the use of at least one electrically conductive or semiconductive polymer as sensitive material in a resistive or gravimetric sensor intended to detect one or more nitro compounds chosen from the group formed by nitroaromatic compounds, nitromines, nitrosamines and nitric esters. Applications include detecting of explosives, checking and monitoring of atmospheric pollution and the quality of the ambient air in relatively confined spaces, monitoring of industrial sites that manufacture, store and/or handle nitro compounds.

### TECHNICAL FIELD

The present invention relates to the use of electrically conductive or semiconductive polymers as sensitive materials for resistive and gravimetric sensors intended for detecting nitro compounds, and in particular nitroaromatic compounds such as nitrobenzene, dinitrobenzene (DNB), dinitrotoluene (DNT), 2,4,6-trinitrotoluene (TNT) and the like.

Such sensors are useful for detecting explosives, whether for the purpose of ensuring security in public places such as airports, for checking the lawfulness of merchandise in circulation in a territory, for combating terrorism, for carrying out disarmament operations, for locating antipersonnel mines or even for decontaminating industrial or military sites.

They are also useful for the protect on of the environment, in particular for checking and monitoring atmospheric pollution and the quality of the ambient air in relatively confined spaces, and also for the monitoring, for security purposes, of industrial sites that manufacture, store and/or handle nitro compounds.

### PRIOR ART

The detection of explosives is a problem of crucial interest, especially as regards civil security.

At the present time, several methods are used for detecting the vapour of nitro compounds found in the composition of explosives, such as the use of “sniffer” dogs trained for this purpose, and the laboratory analysis, for example by chromatography coupled to a mass spectrometer or to an electron capture detector, of samples taken on site, or even infrared detection.

These methods prove in general to be very sensitive, this being of paramount importance for the detection of explosives, on account of the very low vapour concentration of nitro compounds near an explosive. However, the methods are not completely satisfactory.

Thus, the use of “sniffer” dogs has the drawback of requiring both the dogs and their masters to undergo lengthy training, and of being unsuitable for prolonged operations because the attention span of dogs is limited.

As regards the other methods, the sheer size of the apparatus that they use, their energy consumption and their operating costs all prevail against the development of detection systems that are easily transportable and autonomous and, consequently, capable of being used on any type of site.

In recent years, great strides have been made in the development of sensors capable of detecting gaseous chemical species in real time. The operation of these sensors is based on the use of a film of a sensitive material, that is to say a material having at least one physical property that is modified when it is in contact with the gaseous molecules sought, which amounts to a system capable of measuring, in real time, any variation in this physical property and thus to prove the presence of the gaseous molecules sought.

There are many advantages of chemical sensors over the aforementioned methods, namely instant results, possibility of miniaturization and, therefore, portability, handleability and substantial autonomy, low manufacturing and operating costs, etc.

However, it is obvious that their performance is extremely variable, depending on the nature of the sensitive material used.

A number of sensitive materials have already been proposed for detecting gaseous nitro compounds, and more particularly nitroaromatic compounds, among which materials mention may be made of porous silicon, plant-derived carbon, polyethylene glycol, amines, cylclodextrins, cavitands and fluorescent polymers (references [1] to [5]).

Now, within the framework of their work on the development of sensors intended more especially for detecting explosives, the inventors have found that the use of conductive or semiconductive polymers as sensitive materials has resulted in resistive and gravimetric sensors that are extremely efficient for detecting nitro compounds.

It is this observation that forms the basis of the invention.

### SUMMARY OF THE INVENTION

The subject of the invention is the use of at least one electrically conductive or semiconductive polymer as sensitive material in a resistive or gravimetric sensor intended to detect one or more nitro compounds chosen from the group formed by nitroaromatic compounds, nitramines, nitrosamines and nitric esters.

In what follows and in the foregoing, what is meant by an electrically “conductive” polymer is a polymer whose electrical conductivity is at least equal to 10² siemens/cm at room temperature, while what is meant by an electrically “semiconductive” polymer is a polymer whose electrical conductivity is between about 10⁻¹⁰ and 10² siemens/cm at room temperature.

According to the invention, the conductive or semiconductive polymer is preferably a conjugated polymer that is chosen from polymers meeting the following formulae (I), (II), (III), (IV) and (V): 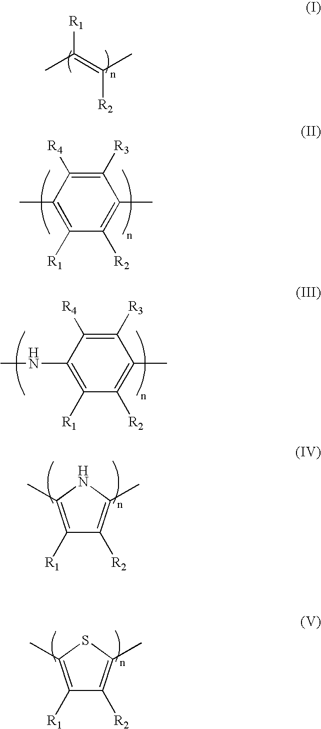
in which n is an integer ranging from 5 to 100 000, while R₁, R₂, R₃ and R₄ represent, independently of one another:

a hydrogen or halogen atom;

a methyl group;

a saturated or unsaturated, linear, branched or cyclic hydrocarbon chain containing 2 to 100 carbon atoms, and optionally one or more heteroatoms and/or one or more chemical functions that include at least one heteroatom, and/or one or more substituted or unsubstituted, aromatic or heteroaromatic groups;

a chemical function that includes at least one heteroatom; or

a substituted or unsubstituted, aromatic or heteroaromatic group.

In the above formulae (I) to (V), when R₁, R₂, R₃ and/or R₄ represent a C₂ to C₁₀₀ hydrocarbon chain and when it includes one or more heteroatoms and/or one or more chemical functions and/or one or more aromatic or heteroaromatic groups, then these atoms, these functions and these groups may form bridges within this chain, or be in side branches of this chain, or may be located at the end of this chain.

The heteroatom or heteroatoms may be any atom other than a carbon or hydrogen atom, for example an oxygen, sulphur, nitrogen, fluorine, chlorine, phosphorus, boron or silicon atom.

The chemical function or functions may especially be chosen from the following functions: —COOH, —COOR₅, —CHO, —CO—, —OH, —OR₅, —SH, —SR₅, —SO₂R₅, —NH₂, —NHR₅, —NR₅R₆, —CONH₂, —CONHR₅, —CONR₅R₆, —C(X)₃, —OC(X)₃, —COX, —CN, —COOCHO and —COOCOR₅ in which:

R₅ represents a saturated or unsaturated, linear, branched or cyclic, hydrocarbon group containing 1 to 100 carbon atoms, or a covalent bond if said chemical function(s) form bridges in a C₂ to C₁₀₀ hydrocarbon chain;

R₆ represents a saturated or unsaturated, linear, branched or cyclic, hydrocarbon group containing 1 to 100 carbon atoms, it being possible for this group to be the same as or different from the hydrocarbon group represented by R₅; while

X represents a halogen atom, for example a fluorine, chlorine or bromine atom.

The aromatic group(s) may be any hydrocarbon group comprising one or more C₃ to C₆ unsaturated rings and including conjugated double bonds such as, for example, a cyclopentadienyl, phenyl, benzyl, biphenyl, phenylacetylenyl, pyrene or anthracene group, whereas the heteroaromatic group(s) may be any aromatic group as defined above, but including, in at least one of its constituent rings, one or more heteroatoms such as, for example, a furanyl, pyrrolyl, thiophenyl, oxazolyl, pyrazolyl, thiazolyl, imidazolyl, triazolyl, pyridinyl, pyranyl, quinoleinyl, pyrazinyl or pyrimidinyl group.

Moreover, in accordance with the invention, this aromatic or heteroaromatic group or these aromatic or heteroaromatic groups may be substituted with one or more chemical functions chosen from the abovementioned functions.

Preferably, the polymer is chosen from polyacetylenes, (polymers of formula (I) in which R₁ and R₂═H), polyphenylenes, (polymers of formula (II) in which R₁ to R₄═H), polyanilines, (polymers of formula (III) in which R₁ to R₄═H), polypyrrols (polymers of formula (IV) in which R₁ and R₂═H), polythiophenes (polymers of formula (V) in which R₁ and R₂═H) and poly(3-alkylthiophenes) (polymers of formula (V) in which R₁═H, whereas R₂═alkyl) and, in particular, is chosen from the latter ones.

As examples of poly(3-alkylthiophenes) that can be used according to the invention, mention may especially be made of poly(3-butylthiophene), poly(3-hexylthiophene), poly(3-octylthiophene), poly(3-decylthiophene) and poly(3-dodecylthiophene).

These polymers may be obtained either from companies selling them—which is for example the case of a number of poly(3-alkylthiophenes) available from Sigma-Aldrich—or by oxidative, enzymatic or electrochemical polymerization of the corresponding monomers or by other such polymerization.

The polyanilines may also be obtained by protonation of emeraldine base, for example by means of an acid, as widely described in the literature.

Moreover, whatever the intrinsic conductivity of these polymers, that is to say the conductivity that they exhibit without any particular treatment, it is possible to subject them to doping and/or dedoping reactions so as to adjust this conductivity to a predetermined value depending on the type of sensor in which they are intended to be used as sensitive material and on the nitro compounds that have to be detected.

These doping reactions may be carried out for example by means of an acid such as hydrochloric acid, camphorsulphonic acid, p-toluenesulphonic acid or 3-nitrobenzenesulphonic acid; of a salt such as iron chloride, nitrosyl trifluoroborate, the sodium salt of anthraquinone-2-sulphonic acid, the sodium salt of 4-octylbenzenesulphonic acid, lithium perchlorate or tetra-n-butylammonium perchlorate; of a halogen such as iodine or bromine; of an alkali metal, such as sodium, potassium or rubidium; and, more generally, by means of any acid or oxidizing agent.

Conversely, the dedoping reactions may be carried out by means of any base or reducing agent, such as ammonia or phenylhydrazine.

The conductivity of a weakly conductive polymer may also be adjusted by adding a polymer having a higher conductivity.

According to the invention, the conductive or semiconductive polymer may also be a polymer that is synthesized in a doped form, that is to say it is obtained by the polymerization of monomers that are chemically bonded beforehand to a doping agent, such as for example aniline monomers each coupled to a molecule of camphorsulphonic acid, or thiophene monomers each coupled to a molecule of a polysulphonate of the polystyrene sulphonate or polyacrylate sulphonate type.

In order for it to be used as sensitive material in a sensor, the conductive or semiconductive polymer is advantageously in the form of a thin film that covers one or both faces of a substrate suitably chosen according to the physical property of the sensitive material the changes in which are intended to be measured by this sensor.

As a variant, the conductive or semiconductive polymer may also be in a bulk form such as, for example, a cylinder having a certain porosity so that all the molecules of the polymer are accessible to the nitro compounds.

When it is in the form of a thin film, this preferably has a thickness ranging from 10 Angstroms to 100 microns.

Such a film may be obtained by any one of the techniques proposed at the present time for producing a thin film on the surface of a substrate, for example:

by spraying, by spin coating or by drop coating a solution containing the conductive or semiconductive polymer onto the substrate;

by dip coating the substrate into a solution containing the conductive or semiconductive polymer;

by the Langmuir-Blodgett technique;

by electrochemical deposition; or

by in situ polymerization, that is to say polymerization directly on the surface of the substrate, of a precursor monomer of the conductive or semiconductive polymer.

However, the inventors have found that the technique used to produce a thin film of a conductive or semiconductive polymer is liable to influence the electrical conductivity of this film and, consequently, the response of the sensor. Thus, for example, thin films of poly(3-alkylthiophenes) produced by spin coating prove to have a very substantially higher electrical conductivity than that of films of similar thickness but obtained by spraying.

Preference will therefore be given to a coating, rather than any other, technique as regards the level of conductivity that it is desired to give the thin film.

The substrate and the measurement system for the sensor are chosen according to the physical property of the sensitive material, the changes in which, induced by the presence of nitro compounds, are intended to be measured by the sensor.

In this case, the changes in two physical properties have proved to be particularly useful to measure. These are, firstly, electrical conductivity changes and, secondly, mass changes.

The sensor is therefore a resistive sensor or a gravimetric sensor.

As examples of gravimetric sensors, mention may be made of quartz microbalance sensors, SAW (surface acoustic wave) sensors, such as Love wave sensors and Lamb wave sensors, and also microlevers.

Among these gravimetric sensors, quartz microbalance sensors are more particularly preferred. This type of sensor, the operating principle of which is described in reference [2], comprises, schematically, a piezoelectric substrate (or resonator), generally a quartz crystal covered on both faces with a metal layer, for example made of gold or platinum, and which is connected to two electrodes. Since the sensitive material covers one or both faces of the substrate, any change in mass of this material is manifested by a change in the vibration frequency of the substrate.

According to the invention, it is possible to combine, within one and the same device or “multisensor” several resistive and/or gravimetric sensors comprising sensitive materials that differ from one another, or are provided with substrate and measurement systems that differ from one another, the essential point being that at least one of these sensors comprises a conductive or semiconductive polymer.

It is also possible to integrate, into such a multisensor, one or more additional sensors comprising, as sensitive material, a conductive or semiconductive polymer as defined above, but designed to measure changes in a physical property other than electrical is conductivity and mass, such as for example changes in an optical property, in particular a fluorescent property. In this case, it is possible either to exploit the fluorescence properties that a certain number of conjugated polymers, including, among others, thiophenes and poly(3-alkylthiophenes), possess naturally or to confer fluorescence properties on the conductive or semiconductive polymer by coupling with an appropriate fluorescent marker.

As mentioned above, the nitro compound(s) intended to be detected by the sensor are chosen from nitroaromatic compounds, nitramines, nitrosamines, and nitric esters, it being possible for these compounds to be in solid, liquid or gaseous (vapour) form.

As examples of nitroaromatic compounds, mention may be made of nitrobenzene, dinitrobenzene, trinitrobenzene, nitrotoluene, dinitrotoluene, trinitrotoluene, dinitrofluorobezene, dinitrotrifluoromethoxybenzene, aminodinitrotoluene, dinitrotrifluoromethylbenzene, chlorodinitrotrifluoromethylbenzene, hexanitrostilbene, trinitrophenylmethylnitramine (or tetryl) and trinitrophenol (or picric acid).

Examples of nitramines are: cyclotetramethylenetetranitramine (or octogen), cyclotrimethylenetrinitramine (or hexogen) and tetryl, whereas an example of a nitrosamine is nitrosodimethylamine.

Examples of nitric esters are: pentrite, ethylene glycol dinitrate, diethylene glycol dinitrate, nitroglycerine and nitroguanidine.

Resistive and gravimetric sensors comprising a conductive or semiconductive polymer as sensitive material, according to the invention, prove to have many advantages, in particular:

an ability to detect nitro compounds with a very high sensitivity since they are capable of detecting their presence in concentrations of the order of ppm, or even lower;

a capability of detecting nitro compounds selectively with respect to other organic compounds, and especially to other aromatic compounds such as toluene;

high speed of response and reproducibility of this response;

capability of operating continuously;

a very satisfactory lifetime, the conductive and semiconductive polymers proving to have good ageing resistance;

a manufacturing cost compatible with the mass-production of sensors, a very small amount of polymer (i.e. a few mg in practice) being necessary to manufacture a sensor; and

the possibility of being miniaturized, and consequently of being able to be easily transported and handled on any type of site.

They are therefore particularly useful for detecting explosives, especially in public places.

Other features and advantages of the invention will become more clearly apparent on reading the rest of the description below, which relates to examples of the use of thin films of conductive or semiconductive polymers in resistive and quartz-microbalance sensors for detecting dinitrotrifluoromethoxybenzene (DNTFMB) vapour and which refer to the appended drawings.

The choice of DNTFMB as nitro compound to be detected was motivated by the fact that this compound is very similar to dinitrotoluene (DNT), this being the nitro derivative most commonly present in the chemical signature of mines based on trinitrotoluene (TNT).

Of course, the examples that follow have been given merely by way of illustrations of the subject matter of the invention and in no way constitute a limitation of this subject matter.

### BRIEF DESCRIPTION OF THE DRAWINGS

FIG. 1 shows the variation in the electrical intensity (curve A) measured across the terminals of a resistive sensor comprising a thin film of undoped polyaniline over the course of an exposure cycle (curve B) during which this sensor is exposed to DNTFMB vapour with a concentration of 3 ppm.

FIG. 2 shows the variation in the electrical intensity (curve A) measured across the terminals of a resistive sensor comprising a thin film of polyaniline doped with camphorsulphonic acid, and then partially dedoped by ammonia vapour, over the course of three exposure cycles (curve B) in which this sensor is exposed to DNTFMB vapour with a concentration of 3 ppm.

FIG. 3 shows the variation in the electrical intensity (curve A) measured across the terminals of a resistive sensor comprising a thin film of poly(3-dodecylthiophene) over the course of three exposure cycles (curve B) in which this sensor is exposed to DNTFMB vapour with a concentration of 3 ppm.

FIG. 4 shows the variation in the vibration frequency (curve A) of the quartz crystal of a quartz microbalance sensor comprising a thin film of poly(3-dodecylthiophene) over the course of two exposure cycles (curve B) in which this sensor is exposed to DNTFMB vapour with a concentration of 3 ppm.

FIG. 5 shows the variation in the electrical intensity measured across the terminals of a resistive sensor comprising a film of poly(3-dodecylthiophene) over the course of two exposure cycles in which this sensor is exposed to DNTFMB vapour followed by four exposure cycles in which it is exposed to dichloromethane, methyl ethyl ketone, toluene and ethanol vapour, respectively.

### EXAMPLES

### Example 1

### Detection of DNTFMB by a Resistive Sensor Comprising a Thin Film of Partially Doped polyaniline

In this example, a resistive sensor comprising an insulating substrate made of alumina provided, on its upper face, with two measurement electrodes formed from interdigitated platinum combs, the width of the tracks being 150 μm, and, on its lower face, with a heating electrode made of platinum was used. That face of the substrate bearing the measurement electrodes was covered with a thin film of partially doped polyaniline.

To do this, a doped polyaniline was firstly prepared by polycondensation of aniline in an oxidizing medium. This polycondensation was carried out by slowly pouring a 100 g/l solution of (NH₄)₂S₂O₈ in 1.5M HCl into a reactor, with stirring, the reactor containing a 100 g/l solution of aniline in 1.5M HCl, and leaving them to react for three days at −40° C. The aniline/oxidizing molar ratio was 1. At the end of the reaction, the mixture was filtered and the polyaniline powder thus recovered was washed in succession with water, with methanol and with ethyl ether.

Next, the polyaniline thus obtained was subjected to partial dedoping by ammonia by stirring, for about 30 minutes, a suspension of polyaniline in methanol and a 1 mole/l ammonia solution.

The mixture was then filtered again and the polyaniline powder washed.

It was then dissolved in N-methylpyrrolidinone with a concentration of 20 g/l.

The polyaniline film was formed on the upper face of the alumina substrate by drop coating, that is to say by depositing on this substrate, three times, two drops of polyaniline solution and by evaporating the N-methylpyrrolidinone after each deposition, by heating to 80° C. A partially doped polyaniline film was thus obtained that passed an electrical intensity of 44 microamps (μA) when a voltage of 1 volt was applied to it.

With a voltage of 1 volt applied to the terminals of the sensor, the latter was subjected to a DNTFMB vapour exposure cycle at room temperature, this cycle comprising a phase in which it was exposed to the ambient air for 600 seconds, then a phase in which it was exposed to DNTFMB with a concentration of 3 ppm for 300 seconds and then again a phase in which it was exposed to the ambient air for 900 seconds.

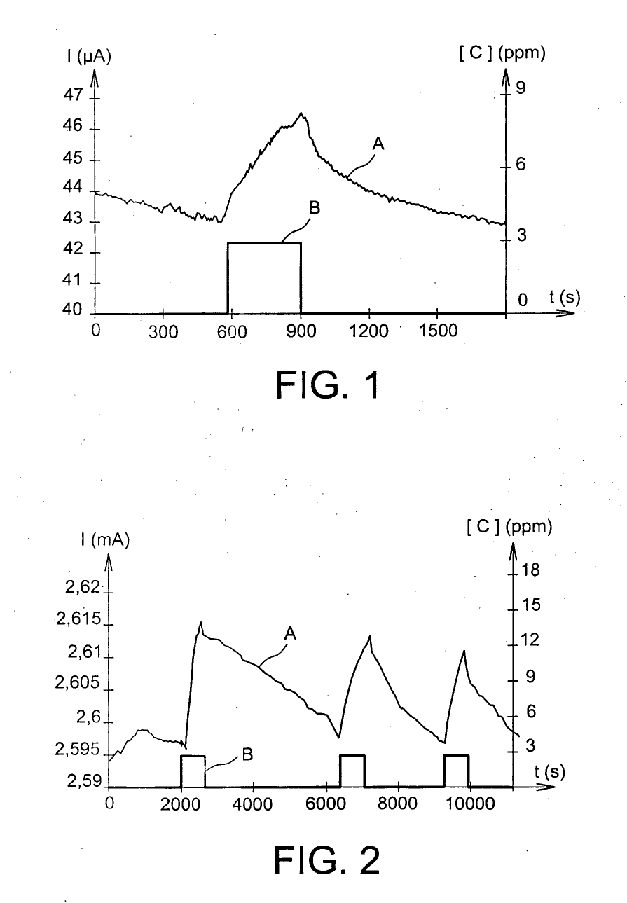

FIG. 1 shows the variation in the electrical intensity measured across the terminals of the sensor over the course of this cycle, curves A and B corresponding to the respective changes in said electrical intensity (I), expressed in μA, and in the. DNTFMB concentration ([C]), expressed in ppm, as a function of time (t), expressed in seconds.

### Example 2

### Detection of DNTFMB by a Resistive Sensor Comprising a Thin Film of Partially Doped polyaniline

In this example, a resistive sensor identical to that used in Example 1 was used, except that the thin film of partially doped polyaniline that covered the upper face of the substrate passed an electrical intensity of 2.6 milliamps (mA) when a voltage of 1 volt was applied to it.

To do this, the upper face of the substrate was firstly covered with a film of polyaniline obtained by polycondensation of aniline in an oxidizing medium, as described in Example 1, and then doped with camphorsulphonic acid, the whole assembly then being exposed to ammonia vapour for 5 minutes in order to partially dedope the polyaniline.

The doping of the polyaniline with camphorsulphonic acid was carried out by mixing the latter with camphorsulphonic acid in a 2/1 molar ratio and then by dissolving this mixture in meta-cresol so as to obtain a 0.3 wt % solution.

The film of doped polyaniline was formed on the upper face of the substrate by drop coating (3 times 2 drops) using a solution containing 1.3 g/l of this polyaniline per litre of meta-cresol.

The ammonia for partially dedoping the polyaniline was generated by heating a 28% solution.

With a voltage of 1 volt applied to the terminals of the sensor, the latter was subjected to three DNTFMB vapour exposure cycles at room temperature:

the first cycle comprising a phase of exposure to the ambient air for 2000 seconds followed by a phase of exposure to DNTFMB for 600 seconds and a phase of exposure to the ambient air for 3900 seconds;

the second cycle comprising a phase of DNTFMB exposure for 600 seconds followed by a phase of exposure to the ambient air for 2300 seconds; and

the third cycle comprising a phase of DNTFMB exposure for 600 seconds followed by a phase of exposure to the ambient air for 1500 seconds, the DNTFMB concentration being 3 ppm in all cases.

FIG. 2 shows the variation in the electrical intensity measured across the terminals of the sensor over the course of these three cycles, curves A and B corresponding to the respective changes in said electrical intensity (I), expressed in mA, and in the DNTFMB concentration ([C]), expressed in ppm, as a function of time (t) expressed in seconds.

### Example 3

### Detection of DNTFMB by a Resistive Sensor Comprising a Thin Film of poly(3-dodecylthiophene)

In this example, a resistive sensor identical to that used in Example 1 was used, except that the upper face of the substrate was covered with a thin film of poly(3-dodecylthiophene), which passed an electrical intensity of 7.4 nanoamps (nA) when a voltage of 1 volt was applied to it.

The poly(3-dodecylthiophene) came from Sigma-Aldrich (reference 450650). It had a molecular mass of 162 000 g/mol.

The poly(3-dodecylthiophene) film was formed on the upper face of the substrate by drop coating (3 times 2 drops) using a solution containing 10 g/l of this polymer per litre of chloroform, the latter being evaporated after each deposition by heating to 45° C.

With a voltage of 1 volt applied to the terminals of the sensor thus obtained, the latter was subjected to three DNTFMB vapour exposure cycles at room temperature:
the first cycle comprising a phase of exposure to the ambient air for 3500 seconds followed by a phase of exposure to the DNTFMB for 600 seconds and then a phase of exposure to the ambient air for 2000 seconds;

the second cycle comprising a phase of exposure to the DNTFMB for 600 seconds followed by a phase of exposure to the ambient air for 2500 seconds; and

the third cycle comprising a phase of exposure to the DNTFMB for 600 seconds followed by a phase of exposure to the ambient air for 4000 seconds, the DNTFMB concentration being 3 ppm in all cases.

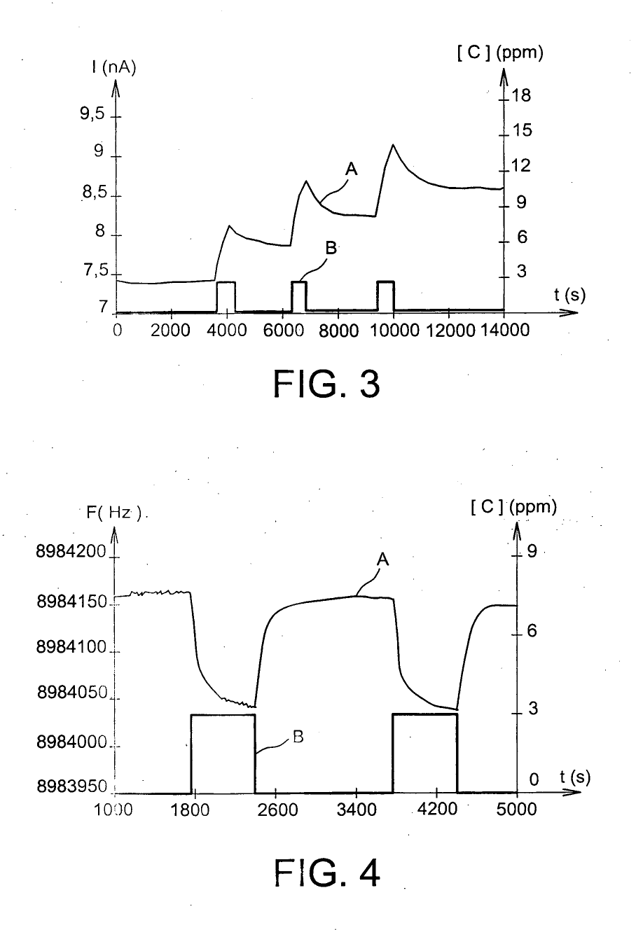

FIG. 3 shows the variation in the electrical intensity measured across the terminals of the sensor over the course of these three cycles, curves A and B corresponding to the respective changes in said electrical intensity (I), expressed in nA, and in the DNTFMB concentration ([C]), expressed in ppm, as a function of time (t) expressed in seconds.

### Example 4

### Detection of DNTFNB by a Quartz Microbalance Sensor Comprising a Thin Film of poly(3-dodecylthiophene)

In this example, a quartz microbalance sensor comprising an AT cut quartz crystal with a vibration frequency of 9 MHz covered with two circular gold measurement electrodes (QA9RA-50 model, Ametek Precision Instruments) was used. The two faces of the device were covered with a thin film of poly(3-dodecylthiophene) with a thickness of about 0.5 μm.

The poly(3-dodecylthiophene) came from Sigma-Aldrich (reference 450650).

The poly(3-dodecylthiophene) film was deposited on each face of the device by spraying a 5 g/l solution of this polymer in chloroform nine times, each lasting 0.4 seconds.

The change in the vibration frequency of the quartz crystal due to this coating was 10.3 kHz.

The sensor was subjected to two DNTFMB vapour exposure cycles at room temperature:

the first cycle comprising a phase of exposure to the ambient air for 750 seconds followed by a phase of exposure to the DNTFMB for 600 seconds and then a phase of exposure to the ambient air for 1400 seconds;

the second cycle comprising a phase of exposure to the DNTFMB for 600 seconds followed by a phase of exposure to the ambient air for 600 seconds;

the DNTFMB concentration being 3 ppm in both cases. FIG. 4 shows the variation in the vibration frequency of the quartz crystal over the course of these two cycles, curves A and B corresponding to the respective changes in the said frequency (F), expressed in Hz, and in the DNTFMB concentration ([C]), expressed in ppm, as a function of time (t) expressed in seconds.

### Example 5

### Study of the Selectivity of a Resistive Sensor Comprising a Film of poly(3-dodecylthiophene)

In this example, a resistive sensor identical to that used in Example 1 was used, except that the upper face of the substrate was covered with a poly(3-dodecylthiophene) film deposited by spraying.

To deposit this coating, 5 mg of poly(3-dodecylthiophene) (Sigma-Aldrich, reference 450650) were dissolved in 1 ml of chloroform and then this solution was sprayed onto the upper face of the substrate nine times each lasting 0.4 seconds, so as to obtain a thin film that passed an electrical intensity of 29 nA when a voltage of 1 volt was applied to it.

With a DC voltage of 1 volt applied to the terminals of the sensor, the latter was subjected to six cycles of exposure to organic compounds in vapour form, comprising:

in the case of the first cycle, a phase of exposure to the ambient air for 1700 seconds, followed by a phase of exposure to the DNTFMB with a concentration of 3 ppm for 600 seconds and a phase of exposure to the ambient air for 5800 seconds;

in the case of the second cycle, a phase of exposure to the DNTFMB with a concentration of 3 ppm for 600 seconds followed by a phase of exposure to the ambient air for 5400 seconds;

in the case of the third cycle, a phase of exposure to dichloromethane with a concentration of 580 000 ppm for 600 seconds followed by a phase of exposure to the ambient air for 520 seconds;

in the case of the fourth cycle, a phase of exposure to methyl ethyl ketone with a concentration of 126 000 ppm for 600 seconds followed by a phase of exposure to the ambient air for 520 seconds;

in the case of the fifth cycle, a phase of exposure to toluene with a concentration of 38 000 ppm for 600 seconds followed by a phase of exposure to the ambient air for 520 seconds; and

in the case of the sixth cycle, a phase of exposure to ethanol with a concentration of 79 000 ppm for 600 seconds followed by a phase of exposure to the ambient air for 280 seconds.

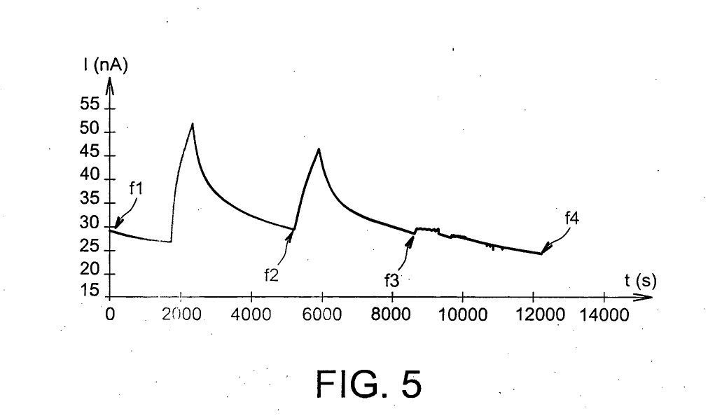

FIG. 5 shows the variation in the electrical intensity (I), expressed in nA, as measured across the terminals of the sensor as a function of time (t) expressed in seconds, the arrow f1 indicating the start of the 1st cycle, the arrow f2 the start of the 2nd cycle, the arrow f3 the start of the 3rd cycle and the arrow f4 the end of the 6th cycle.

### Example 6

### Influence on the Level of Electrical Conductivity of a Resistive Sensor Comprising a Thin Film of poly(3-octylthiophene) or poly(3-dodecylthiophene) of the Technique Used to Produce This Film

In this example, four resistive sensors identical to that used in Example 1 were used, except that the upper face of the substrate of these sensors was covered with a thin film of either poly(3-dodecylthiophene) or poly(3-octylthiophene), this film being produced either by spraying or by spin coating.

The poly(3-dodecylthiophene) and the poly(3-octylthiophene) both came from Sigma-Aldrich.

To produce the thin films by spraying, 5 mg of polymer were dissolved in 1 ml of chloroform and then this solution was sprayed nine times, each lasting 0.4 seconds, onto the upper face of the substrate.

To produce the films by spin coating, 40 mg of polymer were dissolved in 1 ml of xylene and then 10 μl of this solution obtained were deposited, twice, on the upper face of the substrate, which was rotated at 750 rpm for one minute, and then at 2000 rpm for one minute. The films obtained had thicknesses ranging from 1.5 to 1.8 μm.

Table 1 below gives the electrical intensities in nA as measured across the terminals of each of the sensors when a voltage of 1 volt was applied to it.

TABLE 1

| Polymer | Spin coating | Spraying |
|---|---|---|
| poly(3-dodecylthiophene) | 447 nA | 34 nA |
| poly(3-octylthiophene) | 1300 nA | 380 nA |

Examples 1 to 4 above show that resistive or gravimetric sensors comprising a conductive or semiconductive polymer allow nitro compounds such as DNTFMB to be detected with a very high sensitivity. They also show that the response of these sensors is reversible, this reversibility appearing, however, to be more rapid with a quartz microbalance sensor.

Example 5 shows that these sensors, by not reacting in the presence of other organic compounds such as dichloromethane, methyl ethyl ketone, toluene and ethanol, also allow selective detection of nitro compounds.

Finally, Example 6 shows that, if the conductive or semiconductive polymer is used in the form of a thin film, the electrical conductivity of this film is liable to vary in substantial proportions depending on the technique employed to produce it. Thus, the invention offers the possibility of adjusting the electrical conductivity of a thin film intended to serve as sensitive material in a resistive sensor by, firstly, the choice of polymer forming this film, secondly the use of doping and/or dedoping reactions and, thirdly, by the technique with which this film is deposited.

### REFERENCES CITED

[1] Content et al., Chem, Eur. J., 6, 2205, 2000.
[2] Sanchez-Pedrono et al., Anal. Chim. Acta, 182, 285, 1986.
[3] Yang et al., Langmuir, 14, 1505, 1998.
[4] Nelli et al., Sens. Actuators B, 13-14, 302, 1993.
[5] Yang et al., J. Am. Chem. Soc., 120, 11864, 1998.

1. Use of at least one electrically conductive or semiconductive polymer as sensitive material in a resistive or gravimetric sensor intended to detect one or more nitro compounds chosen from the group formed by nitroaromatic compounds, nitramines, nitrosamines and nitric esters.

2. Use according to claim 1, in which the polymer is chosen from polymers meeting the following formulae (I), (II), (III), (IV) and (V): 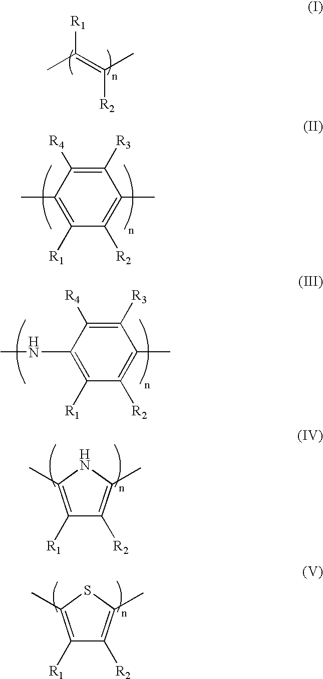
in which n is an integer ranging from 5 to 100 000, while R₁, R₂, R₃ and R₄ represent, independently of one another:
a hydrogen or halogen atom;
a methyl group;
a saturated or unsaturated, linear, branched or cyclic hydrocarbon chain containing 2 to 100 carbon atoms, and optionally one or more heteroatoms and/or one or more chemical functions that include at least one heteroatom, and/or one or more substituted or unsubstituted, aromatic or heteroaromatic groups;
a chemical function that includes at least one heteroatom; or
a substituted or unsubstituted, aromatic or heteroaromatic group.

3. Use according to claim 1, in which the polymer is chosen from polyacetylenes, polyphenylenes, polyanilines, polypyrrols, polythiophenes, and poly(3-alkylthiophenes).

4. Use according to claim 3, in which the polymer is a poly(3-alkylthipohene), in particular a poly(3-dodecylthiophene).

5. Use according to claim 1, in which the polymer is subjected to a doping reaction and/or a dedoping reaction.

6. Use according to claim 1, in which the polymer is used in the sensor in the form of a thin film covering one or both faces of a substrate.

7. Use according to claim 6, in which the thin film measures 10 Angstroms to 100 microns in thickness.

8. Use according to claim 6, in which the thin film is prepared by a technique chosen from spraying, spin coating, drop coating, dip coating, the Langmuir-Blodgett technique, electrochemical deposition and in situ polymerization of a precursor monomer of the polymer.

9. Use according to claim 1, in which the sensor is a quartz microbalance sensor.

10. Use according to claim 1, in which the sensor is a multisensor comprising several sensors that are chosen from resistive and gravimetric sensors, at least one of these sensors comprising an electrically conductive or semiconductive polymer as sensitive material.

11. Use according to claim 1, in which the nitro compound(s) to be detected are in solid, liquid or gaseous form.

12. Use according to claim 1, in which the nitro compound(s) to be detected are chosen from nitrobenzene, dinitrobenzene, trinitrobenzene, nitrotoluene, dinitrofluorobezene, trinitrotoluene, dinitrofluorobezene, dinitrotrifluoromethoxybenzene, dinitrotrifluoromethoxybenzene, aminodinitrotoluene, dinitrotrifluoromethylbenzene, chlorodinitrotrifluoromethylbenzene, hexanitrostilbene, trinitrophenylmethylnitramine and trinitrophenol.

13. Use according to claim 1, for the detection of explosives.
